(19) United States
(12) Patent Application Publication
(10) Pub. No.: US 2026/0258469 A1
(43) Pub. Date: Sep. 3, 2026

(54) PROTEIN PRODUCTION METHOD, CULTURED MEAT PRODUCTION METHOD, ADDITIVE USED FOR CULTURED MEAT PRODUCTION METHOD, AND KIT USED FOR PROTEIN PRODUCTION METHOD

(71) Applicant: NUProtein Co., Ltd., Hyogo (JP)

(72) Inventors: Tomotaka ITAYA, Tokushima (JP); Hiroaki TADA, Tokushima (JP); Masataka MINAMI, Hyogo (JP)

(21) Appl. No.: 18/865,947

(22) PCT Filed: Jan. 15, 2024
(86) PCT No.: PCT/JP2024/000839

§ 371 Date: Nov. 14, 2024

(30) Foreign Application Priority Data
Jun. 5, 2023 (JP) 2023-092204

(51) Int. Cl. C12P 21/02; A23L 13/00

(52) U.S. Cl. CPC C12P 21/02; A23L 13/00

(57) ABSTRACT

An object is to provide a protein production method that causes an energy regeneration enzyme to self-proliferate. The object can be achieved by a protein production method including a protein translation step of translating a protein by using translation template mRNA in the presence of an element for translating translation template mRNA into a protein in the absence of cells, and the translation template mRNA includes translation template mRNA for translating an energy regeneration enzyme.

### TECHNICAL FIELD

The disclosure in the present application relates to a protein production method, a cultured meat production method, an additive used for a cultured meat production method, and a kit used for a protein production method.

### BACKGROUND ART

Synthetic systems for cell-free synthesis of proteins are systems for performing protein synthesis in cell-free by preparing a medium containing intracellular elements related to protein synthesis. Various types of such cell-free protein synthetic systems are known. As such synthetic systems, there are a system for applying template DNA, which is a transcription template, to a medium to synthesize a protein, which is the final product, and a system for applying mRNA, which is a translation template, to a medium to synthesize a protein.

In cell-free protein synthetic systems, systems using extracted liquids prepared from E. coli, insect cells, wheat germ, animal cells, and the like are known, and systems provided as kits are commercially available from several companies. Proteins are synthesized using mRNA as translation templates and using energy source such as ATP or GTP. However, when the energy source is consumed by protein synthesis, ATP changes to AMP or ADP, and GTP changes to GDP, which leads to lack of energy source for protein synthesis. Thus, in cell-free protein synthetic systems, it is known to add an energy regeneration enzyme such as enzymes regenerating AMP or ADP into ATP or enzymes regenerating GDP into GTP. As energy regeneration enzymes, for example, Patent Literature 1 discloses creatine kinase, myokinase, nucleoside diphosphate kinase (NDK), or the like.

If the amount of an energy regeneration enzyme added to a cell-free protein synthetic system is excessively small, the regenerated energy source will also be reduced, and as a result, the amount of synthesis of proteins will also be smaller. It is thus necessary to add a predetermined amount of an energy regeneration enzyme to a cell-free protein synthetic system. For example, Patent Literature 2 discloses using creatine kinase at a concentration of 0.1 mg/mL to 0.5 mg/mL.

### CITATION LIST

### Patent Literature

Patent Literature 1: Japanese Patent No. 4061043
Patent Literature 2: Japanese Patent Application Laid-Open No. 2013-158342

### SUMMARY OF INVENTION

### Technical Problem

Among energy regeneration enzymes, for example, those derived from rabbit muscles are known for creatine kinase, and those derived from enzymes are known for myokinase. However, since the energy regeneration enzymes are of biological origin, there is a problem of needing a step of isolating the enzymes resulting in relatively high prices. Further, to avoid deactivation of the energy regeneration enzymes, there is a problem of needing to be careful about addition of a stabilizing agent, control of the storage temperature, and the like.

The disclosure in the present application has been made in order to solve the above problems of the conventional art. According to an intensive study made by the present inventors, it has been newly found that, (1) by using translation template mRNA that is for translating an energy regeneration enzyme and (2) by causing the energy regeneration enzyme to self-proliferate in accordance with a cell-free protein synthetic system, it is possible to construct an energy regeneration system without adding the energy regeneration enzyme itself isolated into the cell-free protein synthetic system.

That is, the disclosure of the present application intends to provide a protein production method for causing an energy regeneration enzyme to self-proliferate, a cultured meat production method, an additive used for a cultured meat production method, and a kit used for a protein production method.

### Solution to Problem

The disclosure of the present application relates to a protein production method, a cultured meat production method, an additive used for a cultured meat production method, and a kit used for a protein production method as illustrated below.

(1) A protein production method including a protein translation step of translating a protein by using translation template mRNA in the presence of an element for translating translation template mRNA into a protein in the absence of cells,
wherein the translation template mRNA includes translation template mRNA for translating an energy regeneration enzyme.

(2) The protein production method according to (1) above, wherein the translation template mRNA further includes translation template mRNA for translating a target protein.

(3) The protein production method according to (2) above, wherein the protein translation step includes
a first protein translation step of translating the energy regeneration enzyme by using the translation template mRNA for translating the energy regeneration enzyme, and
a second protein translation step of translating the target protein by using a translation product of the first protein translation step and the translation template mRNA for translating the target protein.

(4) The protein production method according to (2) above, wherein the protein translation step is performed in a state where the translation template mRNA for translating the energy regeneration enzyme and the translation template mRNA for translating the target protein coexist.

(5) The protein production method according to any one of (1) to (4) above, wherein the energy regeneration enzyme is at least one type selected from a group consisting of creatine kinase, nucleoside 2-phosphate kinase, arginine kinase, and adenylate kinase.

(6) The protein production method according to any one of (1) to (4) above, wherein the translation template mRNA further includes translation template mRNA for translating at least one type selected from a group consisting of inorganic diphosphatase, inorganic pyrophosphatase, and disulfide isomerase.

(7) The protein production method according to (2) above, wherein the translation template mRNA for translating the energy regeneration enzyme and the translation template mRNA for translating the target protein are translated by
using transcription template DNA including a region to code the translation template mRNA and a promotor region arranged at the 5′ terminal of the region to code the translation template mRNA, and
using the transcription template DNA in the presence of an element for transcribing the transcription template DNA into mRNA in the absence of cells.

(8) The protein production method according to (2) above, wherein the energy regeneration enzyme and the target protein are from the same biological species.

(9) The protein production method according to any one of (2) to (4), (7), and (8) above, wherein the target protein is a growth factor.

(10) A cultured meat production method including a step of culturing cells by using a culture liquid containing a translation product as an additive, the translation product being produced by the protein production method according to (9) above.

(11) An additive used for a cultured meat production method, the additive containing a translation product produced by the protein production method according to (9) above.

(12) A kit used for a protein production method, the kit including:
translation template mRNA for translating an energy regeneration enzyme; and
an element for translating, into a protein, the translation template mRNA for translating the energy regeneration enzyme in the absence of cell.

(13) The kit according to (12) above further including translation template mRNA for translating a target protein.

(14) The kit according to (13) above, wherein the energy regeneration enzyme and the target protein are from the same biological species.

### Advantageous Effects of Invention

The protein production method, the cultured meat production method, the additive used for a cultured meat production method, and the kit used for the protein production method disclosed in the present application can construct an energy regeneration system by using an energy regeneration enzyme translated from a translation template in accordance with a cell-free protein synthetic system. Therefore, it is not required to add an isolated energy regeneration enzyme to a cell-free protein synthetic system.

### BRIEF DESCRIPTION OF DRAWINGS

FIG. 1 is a graph illustrating a temporal change of GFP synthesized in <Reference Example 1>.

FIG. 2 is a diagram illustrating an overview of transcription template DNA for CK.

FIG. 3 is a diagram illustrating an overview of a procedure of preparing CK_DNA by PCR.

FIG. 4 is a photograph substituted for a drawing, which is a Western blot illustrating results of self-proliferation of an energy regeneration enzyme (CK) using a cell-free protein synthetic system in Example 1.

FIG. 5 is a photograph substituted for a drawing, which is a Western blot illustrating results of self-proliferation of an energy regeneration enzyme (CK) of various animals using a cell-free protein synthetic system in Example 2.

FIG. 6 is a graph illustrating results of synthesis of GFP in Example 3 by using the translation product obtained in Example 1

FIG. 7 is a diagram illustrating an overview of transcription template DNA for FGF2 of chicken, Thunnus, and eel.

FIG. 8 is a diagram illustrating an overview of transcription template DNA for FGF2 of bovine.

FIG. 9 is a diagram illustrating an overview of a procedure of preparing FGF2_DNA by PCR.

FIG. 10 is a Western blot illustrating results of synthesis of a target protein (FGF2) by using an energy regeneration enzyme derived from the same biological species in Example 4.

FIG. 11 is a graph illustrating that an energy regeneration system can be self-constructed and a target protein can be synthesized in a state where translation template mRNA for translating an energy regeneration enzyme and translation template mRNA for translating the target protein co-exist in Example 5.

### DESCRIPTION OF EMBODIMENTS

A protein production method, a cultured meat production method, an additive used for a cultured meat production method, and a kit used for a protein production method disclosed in the present application will be described below in detail. Note that the following description is provided for easier understanding, and the scope of technical features disclosed in the present application is not limited to that described below. It is apparent that the disclosure can be changed as appropriate into examples other than the examples below within the scope not compromising the spirit disclosed in the present application.

### First Embodiment of Protein Production Method

The protein production method according to the first embodiment includes a protein translation step of translating a protein by using translation template mRNA in the presence of an element for translating translation template mRNA into a protein in the absence of cells. Further, the translation template mRNA includes translation template mRNA that is for translating an energy regeneration enzyme that is a protein.

Cell-free protein synthetic systems correspond to a method of synthesizing a protein in a test tube by adding translation template mRNA or transcription template DNA to a solution containing amino acids, energy molecules such as ATP or GTP, energy regeneration systems, salts such as magnesium ions, or the like for a cell-free extract liquid containing translation components such as ribosomes, tRNA, aminoacyl-tRNA synthetases, translation initiation factors, translation elongation factors, translation termination factors, or the like. The protein production method disclosed in the present application is to add translation template mRNA that is for translating an energy regeneration enzyme instead of adding an isolated energy regeneration enzyme (separately manufactured as an additive) into a cell-free protein synthetic system. Further, the protein production method disclosed in the present application is characterized in self-constructing an energy regeneration system by using energy regeneration enzyme translated from translation template mRNA in accordance with a cell-free protein synthetic system. Therefore, when “an element for translating translation template mRNA into a protein in the absence of cells” is stated in the present specification, “element” means a solution (translation liquid) containing a cell-free extract liquid, an amino acid, an energy molecule, and salts. Further, “an element for translating translation template mRNA into a protein in the absence of cells” can also be said to be an element “to which an energy regeneration enzyme isolated is not added before and at start of protein synthesis”. The energy regeneration enzymes are typically isolated from animals. Therefore, the expression “an energy regeneration enzyme isolated is not added before and at start of protein synthesis” may be replaced with the expression “an energy regeneration enzyme derived from an animal isolated is not added before and at start of protein synthesis”. Note that, although the protein production method disclosed in the present application is characterized in self-constructing an energy regeneration system by using an energy regeneration enzyme translated from translation template mRNA in accordance with a cell-free protein synthetic system as described above, the addition of an isolated energy regeneration enzyme derived from an animal does not inhibit self-construction of an energy regeneration system from a technical point of view. Therefore, for smooth progress of the initial stage cycle to self-construct an energy regeneration system, addition of a significantly small amount of the isolated energy regeneration enzyme is not excluded. Note that, when adding an isolated energy regeneration enzyme, it is desirable to add the same enzyme as the energy regeneration enzyme translated from translation template mRNA so as not to cause the added energy regeneration enzyme to be handled as an impurity.

As described above, the cell-free protein synthetic system disclosed in the present application is characterized in that the energy regeneration enzyme isolated is not added before and at start of synthesis. The creatine kinase, which is an example of energy regeneration enzymes, mainly exists in muscles of animals. Thus, when a cell-free extract liquid derived from animal cells is used, creatine kinase may be contained in the cell-free extract liquid. Therefore, the cell-free extract liquid disclosed in the present application is preferably a non-animal-based cell-free extract liquid. The non-animal-based cell-free extract liquid may be, but is not limited to, for example, those extracted from wheat germs or the like by a known method.

As the amino acid, various natural amino acids may be used, but non-natural amino acids may be used.

As the energy molecule, known molecules used in protein synthesis may be used. The energy molecule may be, for example, nucleotide triphosphate, creatine phosphate, and formyl folate. Herein, nucleotide triphosphate may be ATP, GTP, CTP, or UTP. When a protein is produced from translation template mRNA, ATP and GTP may be used as the energy molecule. When a protein is produced from transcription template DNA, ATP, GTP, CTP, and UTP may be used.

The energy regeneration enzyme may be known enzymes in the field of protein synthesis. Although not limited, the energy regeneration enzyme may be, for example, creatine kinase, nucleoside 2-phosphate kinase, arginine kinase, pyruvate kinase, adenylate kinase, and the like.

Creatine kinase catalyzes a reaction between creatine phosphate and AMP and regenerates ADP into ATP. Nucleoside 2-phosphate kinase regenerates AMP and ADP into ATP and regenerates GDP into GTP in which a polyphosphoric acid is used as a substrate. Arginine kinase phosphorylates arginine (Arg) to produce arginine phosphate (PArg) and transfers a phosphoric acid of produced PArg to ADP to regenerate ATP. Pyruvate kinase transfers a phosphate group from phosphoenolpyruvic acid to ADP to regenerate ATP. Adenylate kinase is an enzyme that produces one molecule of AMP and one molecule of ATP from two molecules of ADP.

The translation template mRNA for translating an energy regeneration enzyme is not particularly limited as long as it can translate the energy regeneration enzymes described above. The translation template mRNA includes a code region to code an amino acid sequence of an energy regeneration enzyme, and untranslated regions can be coupled to both the 5′ and 3′ sides of the code region as appropriate. Examples of the 5′ untranslated region and the 3′ untranslated region for improving the translation efficiency of a protein are disclosed in International Publication No. 2022/185664 and International Publication No. 2021/070616 filed by the present applicant. For the translation template mRNA disclosed in the present application, the untranslated region disclosed in International Publication No. 2022/185664 and International Publication No. 2021/070616 may be used as needed. The features disclosed in International Publication No. 2022/185664 and International Publication No. 2021/070616 are incorporated in the present specification by reference.

As the salts, known salts used in cell-free protein synthetic systems can be used. The salts may be, but are not limited to, for example, magnesium acetate, potassium acetate, calcium chloride, or the like.

The protein production method according to the first embodiment achieves the following advantageous effects.

(1) No energy regeneration enzyme is added before and at start of protein synthesis, and an energy regeneration system is constructed by using only the energy regeneration enzyme translated from translation template mRNA that is for translating the energy regeneration enzyme. Therefore, since the isolated energy regeneration enzyme is not required, costs can be reduced.

(2) The protein produced in accordance with the cell-free protein synthetic system can be used as pharmaceuticals and foods. However, when a protein derived from a biological species that has never been taken in is taken in as a pharmaceutical or a food, there is a risk of an allergic reaction or the like. For example, choices of biological species from which a commercially available energy regeneration enzyme is derived are limited, such as creatine kinase being derived from rabbits. On the other hand, since energy regeneration enzymes are obtained by being translated from translation template mRNA in the present application, an energy regeneration enzyme derived from a desired biological species can be translated. Therefore, the risk of allergy or the like can be reduced.

(3) When produced proteins are used for foods or pharmaceuticals, some raw materials from a particular biological species may be unable to be used because of a need for avoiding religious reasons or contamination with zoonotic viruses. In the protein production method of the present application, however, an energy regeneration enzyme derived from a desired biological species can be obtained via translation, and this makes it possible to solve the problem due to religious regions or contamination with zoonotic viruses.

(4) Due to influences of prion disease or the like, the use of raw materials derived from animals to manufacture foods or pharmaceuticals tends to be avoided. In the protein production method of the present application, proteins can be produced by using only non-animal raw materials even when the energy regeneration enzymes are derived from animal species. Therefore, there is no risk of disease or the like derived from animal raw materials.

### Second Embodiment of Protein Production Method

Next, a second embodiment of a protein production method will be described. The protein production method according to the second embodiment differs from the protein production method according to the first embodiment in that translation template mRNA for translating a target protein is included, and other features are the same as those of the protein production method according to the first embodiment. To avoid duplicated description, in the second embodiment of the protein production method, different features from those of the first embodiment will be mainly described. It is therefore apparent that, although not explicitly described in the second embodiment, features that have already been described in the first embodiment can be applied to the second embodiment.

The target protein is not particularly limited as long as it is a protein that can be synthesized in accordance with a cell-free protein synthetic system. The target protein may be, for example, growth factors; secreted proteins such as insulin or amylase or plasma proteins such as transferrin, myoglobin, or albumin; or the like. Further, the animal species of the target protein may be, for example, fish such as salmons, eels, or the like; mammals such as human, bovine, rabbit, or the like; echinoderms such as sea urchins; or the like.

The protein translation step of the protein production method according to the second embodiment is not particularly limited as long as it can produce a target protein by using an energy regeneration enzyme translated from translation template mRNA that is for translating an energy regeneration enzyme.

An example of the protein translation step may be the following example (hereafter, which may be referred to as “first example”):
first, performing a first protein translation step of translating an energy regeneration enzyme by using translation template mRNA that is for translating the energy regeneration enzyme; and
next, performing a second protein translation step of translating a target protein by using a translation product of the first protein translation step and translation template mRNA that is for translating the target protein.

Another example of the protein translation step may be the following example (hereafter, which may be referred to as “second example”):
performing the protein translation step in a state where translation template mRNA for translating an energy regeneration enzyme and translation template mRNA for translating a target protein coexist.

In the case of the first example, since the energy regeneration enzyme is first translated from the translation template mRNA in the first protein translation step, it is possible to adjust the amount of the energy regeneration enzyme to be translated by adjusting the reaction time. Further, it is also possible to adjust the amount of the translation product in the first protein translation step to be added when performing the second protein translation step. Therefore, in the case of the first example, it is possible to adjust the amount of the energy regeneration enzyme contained in the cell-free protein synthetic system when translating the target protein. With the adjustment of the amount of the energy regeneration enzyme, an advantageous effect that the translation efficiency of the target protein can be optimized is achieved. On the other hand, in the case of the second example, although the translation efficiency of the target protein is lower than that of the first example because the translation of the energy regeneration enzyme and the translation of the target protein are performed at the same time, convenience of protein production is improved because there is no need for dividing the protein translation step.

The translation template mRNA for translating the energy regeneration enzyme and the target protein may be translation template mRNA synthesized by a nucleic acid synthesis device or may be translation template mRNA transcribed from transcription template DNA. The transcription template DNA can include a region to code translation template mRNA and a promotor sequence arranged at the 5′ terminal of the region to code the translation template mRNA. The translation template mRNA can be synthesized by being translated using transcription template DNA in the presence of an element for transcribing transcription template DNA into mRNA in the absence of cells. For the promotor sequence and the element for transcribing transcription template DNA into mRNA, those known in this technical field can be used. The promotor sequence may be T7 promotor sequence, SP6 promotor sequence, T3 promotor sequence, or the like, which are examples without limitation.

The protein production method according to the second embodiment achieves the following advantageous effect (5) in addition to the advantageous effects described in (1) to (4) achieved by the protein production method according to the first embodiment.

(5) It achieves an advantageous effect obtained when the term “energy regeneration enzyme” is replaced with the term “target protein” in the advantageous effects described in (2) to (4) achieved by the protein production method according to the first embodiment.

Optional and additional features that can be employed in the embodiments of the protein production method

Next, optional and additional features that can be employed in the embodiments of the protein production method will be described.

<Addition of Translation Template mRNA to Translate Other Enzymes>

In the protein translation step, translation template mRNA for translating an enzyme that is for decomposing a byproduct (for example, inorganic pyrophosphoric acid) generated in transcription/translation and/or an enzyme that is for folding a target protein (activating a target protein) may be added in addition to the energy regeneration enzyme. The enzyme for decomposing a byproduct may be, but is not limited to, for example, inorganic diphosphatase, inorganic pyrophosphatase, or the like. Further, the enzyme for folding a target protein may be, but is not limited to, for example, disulfide isomerase or the like.

When the protein translation step employs the first example, the translation template mRNA for translating an enzyme that is for decomposing a byproduct and/or an enzyme that is for folding a target protein may be added in the first protein translation step, may be added in the second protein translation step, or may be added in both the first protein translation step and the second protein translation step. When the protein translation step employs the second example, the translation template mRNA for translating the enzyme that is for decomposing a byproduct and/or the enzyme that is for folding a target protein can be added together with the translation template mRNA for translating an energy regeneration enzyme and the translation template mRNA for translating a target protein. When translation template mRNA to translate other enzymes is prepared by being transcribed from the transcription template DNA, transcription template DNA including a region to code the translation template mRNA and the promotor region arranged at the 5′ terminal of the region to code the translation template mRNA can be designed. In the protein translation step, the enzyme for decomposing a byproduct and/or the enzyme for folding a target protein coexist, and thus, the translation efficiency of the target protein is improved. Note that the enzyme for decomposing a byproduct and/or the enzyme for folding a target protein are mere preferable examples and may be any other enzymes that improve the translation efficiency of a target protein.

When the translation template mRNA to translate other enzymes is added, the following advantageous effect (6) is achieved in addition to the advantageous effects achieved by the first and second embodiments of the protein production method.

(6) Since a byproduct that would inhibit efficient synthesis of a cell-free protein synthetic system can be removed, the protein production efficiency is improved. Further, the produced protein can be activated.

### <Biological Species of Energy Regeneration Enzyme and Target Protein>

The energy regeneration enzyme and the target protein in combination may be derived from the same biological species. In such a case, the nucleic acid sequences of an energy regeneration enzyme and a target protein of the same biological species can be obtained from a known DB to design translation template mRNA. Further, when the translation template mRNA is prepared from transcription from transcription template DNA, transcription template DNA including a region to code the translation template mRNA and a promotor region arranged at the 5′ terminal of the region to code the translation template mRNA can be designed.

When the energy regeneration enzyme and the target protein in combination are derived from the same biological species, the following advantageous effect (7) is achieved in addition to the advantageous effects achieved by the first and second embodiments of the protein production method.

(7) When the produced protein is used for foods, mixing of proteins derived from different biological species is avoided in some occasions. Because the energy regeneration enzyme and the target protein in combination are derived from the same biological species, a likelihood of needing such avoidance is reduced.

### <Target Protein>

The target protein may be a growth factor or a cytokine. In recent years, cultured meats have attracted attention in terms of measures to cope with population growth and increased demand for food production, environmental problems due to an increase of pastures for feeding animals used for meat, safety against administration of antibiotics to animals, ethics against taking animal lives, or the like. While stem cells taken out from animals are cultured into cultured meats by using a culture liquid containing substances necessary for growth of cells, such as amino acids or carbon hydrates, growth factors are required for the culturing. Further, cytokines are required for cell differentiation. The growth factor or the cytokine may be, but is not limited to, for example, the following growth factors.
Epidermal growth factor (EGF)
Insulin-like growth factor (IGF)
Transforming growth factor (TGF)
basic fibroblast growth factor (bFGF or FGF2)
Nerve growth factor (NGF)
Brain-derived neurotrophic factor (BDNF)
Vesicular endothelial growth factor (VEGF)
Granulocyte-colony stimulating factor (G-CSF)
Granulocyte-macrophage-colony stimulating factor (GM-CSF)
Platelet-derived growth factor (PDGF)
Erythropoietin (EPO)
Thrombopoietin (TPO)
Hepatocyte growth factor (HGF)
leukemia inhibitory factor (LIF)

When the target protein is a growth factor, the following advantageous effect (8) is achieved in addition to the advantageous effects achieved by the first and second embodiments of the protein production method.

(8) Growth factors are very expensive. However, with the use of the protein production method disclosed in the present application, growth factors can be inexpensively manufactured.

Although the protein production method disclosed in the present application has been described above with the specific embodiments and several optional and additional features that can be employed, various modifications may be made as long as they are within the scope of the technical concept disclosed in the present application without being limited to these embodiments. Further, the optional and additional features illustrated as examples that can be employed may be added alone or may be added in combination of any two or more of the additional features. When any two or more of the additional features are used in combination, respective effects are synergistically achieved. Further, if necessary, a sequence for adding a protein tag on the N-terminal side and/or the C-terminal side of the energy regeneration enzyme and/or the target protein may be coupled to translation template mRNA or transcription template DNA.

### Embodiment of Cultured Meat Production Method and Embodiment of Additive

In the protein production method, when growth factors are produced as the target protein, the translation product can be used for a cultured meat production method. The cultured meat production method can be performed by a cell culturing step by using a culture liquid containing a translation product as an additive. For the culture liquid, any known culture liquid can be used. For the cells, any cells of animals to be cultured can be used as described above. The animals to be cultured are not particularly limited as long as they are taken in as edible meat. The animals may be, but are not limited to, mammals such as bovine, pig, horse, or sheep; birds such as chicken, duck, or quail; fish such as salmon, eel, or Thunnus; crustaceans such as shrimp or crab; or the like.

Further, in the protein production method, when a growth factor is produced as the target protein, the translation product can be used as the additive used for the cultured meat production method.

The embodiment of the cultured meat production method and the embodiment of the additive achieve the following advantageous effects.

(9) Since the growth factor can be inexpensively available, the cost of cultured meat can be reduced.

(10) When an energy regeneration enzyme and a growth factor from the same biological species as the animal to be cultured are used, a cultured meat that does not contain a protein derived from other types of animals can be produced.

### Embodiment of Kit Used for Protein Production Method

Next, an embodiment of a kit used for a protein production method will be described. As described above, in the conventional cell-free protein synthetic systems, enzymes isolated from animals or the like are used for an energy regeneration enzyme. In contrast, it has been newly found by the present inventors that, in cell-free protein synthetic systems, an energy regeneration system is constructed by using an energy regeneration enzyme obtained from translation template mRNA that is for translating the energy regeneration enzyme. Therefore, a kit used for a protein production method including translation template mRNA for translating an energy regeneration enzyme and an element for translating translation template mRNA into a protein in the absence of cells is a novel invention. Note that, in the kit used for the protein production method according to the embodiment, since only the energy regeneration enzyme translated from translation template mRNA for translating the energy regeneration enzyme is used to construct an energy regeneration system, the kit can be said to be a kit to which no energy regeneration enzyme is added. Further, the kit may further include translation template mRNA for translating a target protein, and the energy regeneration enzyme and the target protein may be from the same biological species.

The terms “translation template mRNA (that is) for translating an energy regeneration enzyme”, “element for translating translation template mRNA into a protein in the absence of cells”, and “translation template mRNA (that is) for translating a target protein” have already been described in the embodiment of the protein production method. Accordingly, the detailed description thereof will be omitted for avoiding duplicated description.

The “translation template mRNA (that is) for translating an energy regeneration enzyme”, “element for translating translation template mRNA into a protein in the absence of cells”, and “translation template mRNA (that is) for translating a target protein” can be separately provided and then mixed with each other in use. Alternatively, the “translation template mRNA (that is) for translating an energy regeneration enzyme” and “element for translating translation template mRNA into a protein in the absence of cells” may be provided mixed with each other, and the “translation template mRNA (that is) for translating a target protein” may be provided as a separate member. Further alternatively, the “translation template mRNA (that is) for translating an energy regeneration enzyme”, “element for translating translation template mRNA into a protein in the absence of cells”, and “translation template mRNA (that is) for translating a target protein” may be provided all mixed with each other.

While the embodiments disclosed in the present application will be specifically described with Examples presented below, these Examples are merely provided for description of the embodiments. The Examples are intended neither to limit the scope of the invention disclosed in the present application nor to express limitation of the same.

### EXAMPLES

### Reference Example 1

Importance of creatine kinase in protein production using cell-free protein synthetic system

First, the difference in protein synthesis between cases with and without using purified creatine kinase from rabbit in cell-free protein synthesis of GFP, which is a fluorescent protein, was observed. The experimental procedure is described below.

### (1) Raw Material

WGE: wheat germ extract liquid, prepared in accordance with the procedure described in Japanese Patent Application Laid-Open No. 2006-288320.
CK: creatine kinase from Roche Diagnostics Deutschland GmbH (Product number: 10127566001)
AAM: amino acid substrate. Amino acid substrate attached to PSS 5100 from NUProtein CO., LTD. was used.
mRNA: designed so as to include a code region to code the amino acid sequence of GFP of water jellyfish. Note that mRNA was obtained by transcribing transcription template DNA in which a sequence (Acc. No. P42212, purchased from Eurofins Genomics) to code GFP was inserted into the portion of CK_Organism of Table 3 in <Example 1> described later, and this transcription was performed in the same procedure as in Example 1.

### (2) Translation Reaction

Translation reaction liquids having the composition listed in Table 1 below were prepared in 1.5 mL tubes. Note that the unit of the composition liquids listed in Table 1 below is μL. The stock concentration of added CK was 20 mg/mL, and the final concentration was 200 ng/μL. The value X in Table 1 is 77.8 for CK+ and 80 for CK−.

TABLE 1

| | CK+ | CK− |
|---|---|---|
| WGE | 20 | 20 |
| CK | 2.2 | 0 |
| AAM | 40 | 40 |
| mRNA (GFP) | 80 | 80 |
| MilliQ | X | X |
| total | 220 | 220 |

The reaction liquids having the composition listed in Table 1 were added onto a 96-well flat bottom titer plate, which were allowed to react in a plate reader at room temperature (about 23° C.).

### Fluorescence Measurement of Synthesized GFP

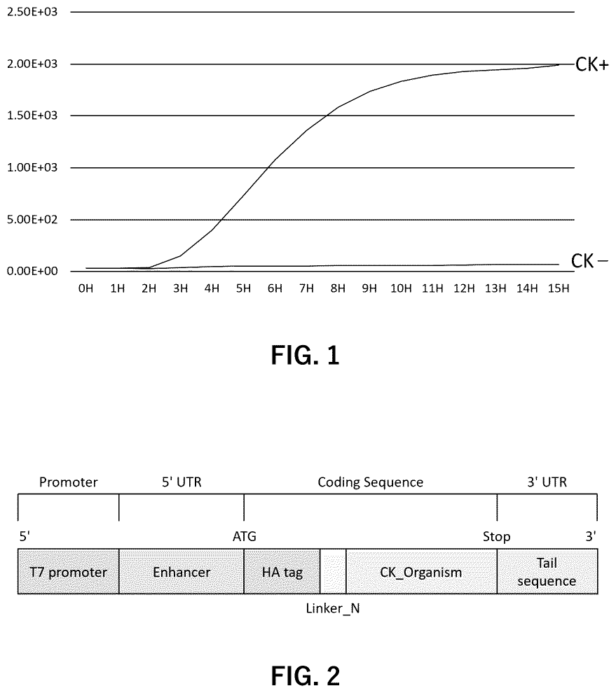

Out of 220 μL of the solution containing GFP synthesized in Reference example 1, 200 μL was used as a sample and irradiated with excitation light having a wavelength of 475 nm for 0.5 seconds, and the fluorescence intensity from GFP was measured by a plate reader every 1 hour for 15 hours (absorption filter of 500 to 550 nm). For the plate reader, GloMax (registered trademark) plate reader (from Promega) was used. FIG. 1 illustrates the measurement results.

As is clear from FIG. 1, with the addition of CK, which is an energy regeneration enzyme, to the cell-free protein synthetic system (CK+), the amount of synthesis of GFP temporarily increased. On the other hand, in the case without the addition of CK (CK−), almost no GFP was synthesized. From the above results, it was confirmed that, when the cell-free protein synthetic system does not have an energy regeneration system, the target protein is not efficiently synthesized.

### Observation of Self-Proliferation of Energy Regeneration Enzyme Using Cell-Free Protein Synthetic System

### Example 1

### (1) Creatine Kinase Sequence

For energy regeneration enzymes derived from various organisms, Table 2 lists sequence information (ACC. No.), biological species (Organism), and suppliers (Company) about creatine kinase (CK). Note that, in Example 1, the sample No. 4, CK_T (Thunnus) was used. The samples Nos. 1 to 3 and 5 were used in Examples 2 to 4 described later.

TABLE 2

| | | Protein | | | |
|---|---|---|---|---|---|
| No. | mark | Name | Acc. No. | Organism | Company |
| 1 | CK_R | Creatine | NM_001082239.1 | Rabbit | GenScript |
| | | Kinase | | | |
| 2 | CK_B | Creatine | NP_777198.2 | Bovine | Eurofins |
| | | Kinase | | | Genomics |
| 3 | CK_C | Creatine | NP_990838.1 | Chicken | Eurofins |
| | | Kinase | | | Genomics |
| 4 | CK_T | Creatine | XP_042286032.1 | Thunnus | Eurofins |
| | | Kinase | | | Genomics |
| 5 | CK_U | Creatine | XP_035288992.1 | Unagi | Eurofins |
| | | Kinase | | | Genomics |

### (2) Transcription Template DNA

FIG. 2 illustrates the overview of the transcription template DNA for CK (hereafter, which may be referred to as “CK_DNA”). Further, Table 3 lists sequences of respective regions of CK_DNA. Note that the CK sequences of Organism in Nos. 1 to 5 described above are inserted in the field for CK_Organism in Table 3 below.

TABLE 3

| Region | Site | DNA Sequence | SEQ.ID. |
|---|---|---|---|
| Promoter | T7 promoter | CCCGCGAAAT TAATACGACT CACTATA | 1 |
| 5′UTR | Enhancer | GGGCTCACCT ATCTCTCTAC ACAAAACATT TCCCTACATA | 2 |
| | | CAACTTTCAA CTTCCTATT | |
| Coding | Start codon | ATG | |
| Sequence | HA tag | TAT CCT TAT GAC GTG CCT GAC TAT GCC | 3 |
| | Linker_N | CTC CAG CAG GGA GGT ACT | 4 |
| | CK_Organism | | |
| | Stop codon | TAG | |
| 3′UTR | Tail sequence | AATAAGTGCT CGGGCGGGCC AAAAAAAAAA AAAAA | 5 |

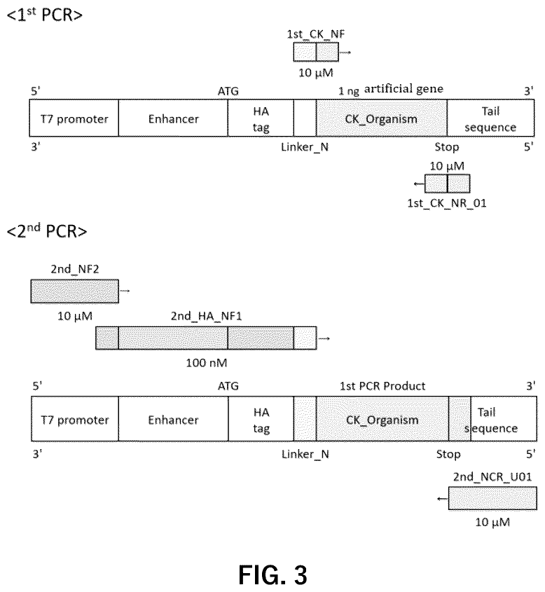

The procedure of preparing CK_DNA by PCR will be described with reference to FIG. 3. For CK_DNA, primers were designed as illustrated in FIG. 3, and CK_DNA was prepared by two-step PCR. Table 4 lists PCR primers, Table 5 and Table 6 list reaction solution compositions for PCR, and Table 7 lists a PCR program. Note that the transcription template DNA synthesis method or the like were performed in accordance with the instruction manual of PSS 5100 (May-2021/Ver. 3.00) from NUProtein CO., LTD. Note that the reagent and the machine used are as follows.
PCR enzyme: KOD-Plus-Neo from TOYOBO CO., LTD.
Primer, artificial gene: Eurofin Genomics, Inc. contract synthesis services
Thermal Cycler: Mastecycler X50s from Eppendorf
High-speed refrigerated micro centrifuge: MX-307 from TOMY KOGYO CO., LTD.

TABLE 4

| Primer name | Primer sequence 5′ to 3′ | SEQ.ID. |
|---|---|---|
| 1st_CK_NF | CCAGCAGGGAGGTACT ATGCCCTTTGGTAACACAC | 6 |
| 1st_CK_NR_01 | GGCCCGCCCGAGCACTTATTCTA CTTTTGAGCGGGTATCATAGAG | 7 |
| 2nd_HA_NF1 | CGACTCACTA TAGGGCTCAC CTATCTCTCT ACACAAAACA | 8 |
| | TTTCCCTACA TACAACTTTC AACTTCCTAT TATGTATCCT | |
| | TATGACGTGC CTGACTATGC CCTCCAGCAG GGAGGTACTA TG | |
| 2nd_NF2 | CCCGCGAAAT TAATACGACT CACTATAG | 9 |
| 2nd_NCR_U01 | TTTTTTTTTT TTTTTGGCCC GCCCGAGCAC | 10 |

TABLE 5
1st PCR Reaction Mixture Composition

| Reagents | Volume |
|---|---|
| 10x PCR buffer | 5 μL |
| 2 mM dNTPs | 5 μL |
| 25 mM MgSO₄ | 3 μL |
| 10 μM 1st_CK_NF | 1 μL |
| 10 μM 1st_CK_NR_01 | 1 μL |
| 1 ng/μL artificial gene | 1 μL |
| PCR DNA Polymerase | 1 μL |
| Ultra pure water | 33 μL |
| Total | 50 μL |

TABLE 6
2nd PCR Reaction Mixture Composition

| Reagents | Volume |
|---|---|
| 10x PCR buffer | 5 μL |
| 2 mM dNTPs | 5 μL |
| 25 mM MgSO₄ | 3 μL |
| 10 μM 2nd_NF2 | 1 μL |
| 100 nM 2nd_HA_NF1 | 1 μL |
| 10 μM 2nd_NCR_U01 | 1 μL |
| 1st PCR product | 1 μL |
| PCR DNA Polymerase | 1 μL |
| Ultra pure water | 32 μL |
| Total | 50 μL |

TABLE 7
1st/2nd PCR Program

| Seg. | Temp. | Time | Cycle |
|---|---|---|---|
| 3 | 94° C. | 2 min | 1 |
| 2-1 | 98° C. | 10 sec | 30 |
| 2-2 | 68° C. | 1 min | |
| 3 | 20° C. | — | — |

### (3) Transcription Reaction

Next, the prepared transcription template DNA was used to prepare translation template mRNA. For the transcription reaction, the following reaction liquid of PSS 5100 from NUProtein CO., LTD. was used, and the reaction liquid was prepared in a 1.5 mL tube by using 2.5 μL of the second PCR reaction solution (containing transcription template DNA) prepared previously and incubated at 37° C. for 3 hours.

TABLE 8

| Reagents | Volume | |
|---|---|---|
| 10x Transcription buffer | 2.5 | μL |
| 25 mM NTP Mix | 2.5 | μL |
| T7 RNA Polymerase | 1 | μL |
| 100 mM DTT | 1.25 | μL |
| 2nd PCR Product | 2.5 | μL |
| Ultra pure water | 15.25 | μL |
| Total | 25 | μL |

To 25 μL of the transcription reaction liquid, 10 μL of 4M ammonium acetate was added and well mixed, and 100 μL of 100% ethanol was further added thereto and mixed, and the resultant was flashed by a desktop centrifuge and then was allowed to stand at −20° C. for 10 minutes. The resultant was then centrifuged (12,000 g, 15 minutes, 4° C.). After the supernatant was removed, the resultant was flashed by using the desktop centrifuge. The supernatant was removed again, and the resultant was allowed to stand until the precipitate dried. Then, 80 μL of ultrapure water was added to 25 μL of the transcription reaction liquid, and the precipitate was dissolved. This was used as the translation template mRNA solution.

### (4) Translation Reaction

Next, the translation reaction liquid having the following composition was prepared in a 1.5 mL tube, put into an incubator set at 23° C., and allowed to react for 15 hours. In the composition listed in Table 9, CK was added in addition to the translation template mRNA for CK_T. In the composition listed in Table 10, only the translation template mRNA for CK_T was added, and no CK was added to the translation reaction liquid. 1. Note that, for comparison, a translation reaction was performed by adding the translation template mRNA for GFP prepared in <Reference Example 1> instead of the translation template mRNA for CK_T of Table 10. Note that the unit of numerical values in Table 9 and Table 10 is μL, the value X in Table 9 is 77.8, and the value X in Table 10 is 80. The stock concentration of the added CK was 20 mg/mL, and the final concentration was 200 ng/μL.

TABLE 9

| | CK+ |
|---|---|
| WGE | 20 |
| CK | 2.2 |
| AAM | 40 |
| mRNA | |
| CK_T | 80 |
| MilliQ | X |
| total | 220 |

TABLE 10

| | CK− |
|---|---|
| WGE | 20 |
| CK | 0 |
| AAM | 40 |
| mRNA | |
| CK_T | 80 |
| MilliQ | X |
| total | 220 |

After the reaction, the 1.5 mL tube was centrifuged (15,000 g, 15 minutes, 4° C.), and the supernatant was used as a protein solution after completion of the translation. The obtained proteins were subjected to Western blotting.

### <Reagents Used>

Gel: 4 to 15% Tris-Glycine gel (from Bio-Rad Laboratories)
membrane: 0.2 μm PVDF membrane (from Bio-Rad Laboratories)
Primary antibody: HA antibody (from Proteintech)
Secondary antibody: goat anti mouse HRP antibody (from Southern Biotech)
Western blot luminescent reagent: SuperSignal West Pico (from Thermo)

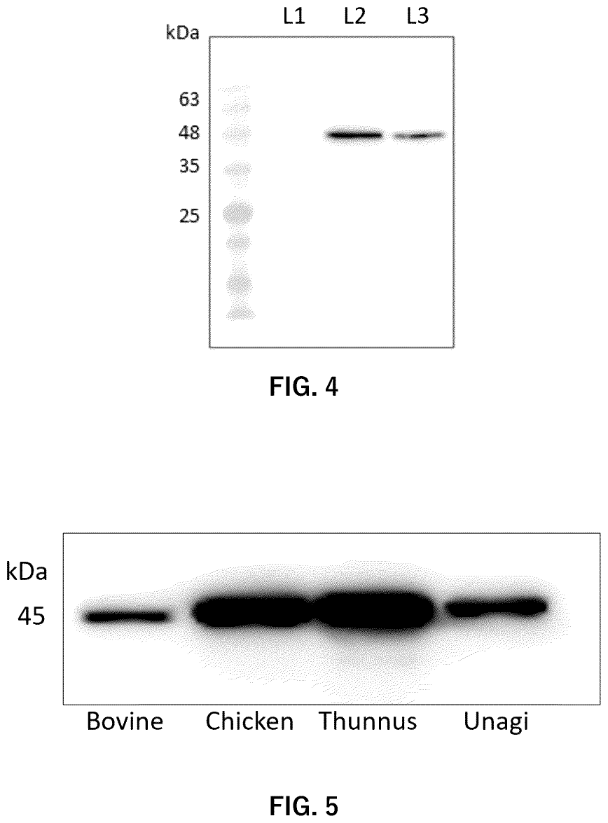

FIG. 4 illustrates the results. In FIG. 4, L2 represents the result about the translation product obtained by the composition listed in Table 9, L3 represents the result about the translation product obtained by the composition listed in Table 10, and L1 represents the result about the translation product obtained by the composition using the translation template mRNA for GFP. As is clear from FIG. 4, it was confirmed that, in L3 in which the isolated CK was not added to the translation reaction liquid, a certain amount of CK_T was translated from the translation template mRNA though the amount is less than that of L2. From the above results, it is considered that, when a translation reaction with the composition listed in Table 10 is performed, CK_T was translated from the translation template mRNA by the element contained in the translation reaction liquid, the translated CK_T constructed an energy regeneration system, and thereby CK_T self-proliferated.

### Example 2

An experiment was performed in the same procedure as in Example 1 except that CK_B (Bovine), CK_C (Chicken), and CK_U (Unagi (eel)) were used instead of CK_T (Thunnus) of Example 1 and that the primers having the following sequences were used as 1st_CK_NF and 1st_CK_NR_01. Note that the primer of CK_R (Rabbit) used in Example 4 described later is listed together. In Table 11, each biological species is denoted after CK, and thereby it is identified which biological species each primer corresponds to.

TABLE 11

| Primer name | Primer sequence 5′ to 3′ | SEQ.ID. |
|---|---|---|
| 1st_CK_Bovine_NF | CCAGCAGGGAGGTACT ATGCCCTTTGGCAATACCCATAAC | 11 |
| 1st_CK_Bovine_NR_01 | GGCCCGCCCGAGCACTTATTCTA CTTTTGGGCTGGGATCA | 12 |
| 1st_CK_Chicken_NF | CCAGCAGGGAGGTACT ATGCCCTTCTCCAGCACAC | 13 |
| 1st_CK_Chicken_NR_01 | GGCCCGCCCGAGCACTTATTCTA CTTTTGCGCTGGAATCA | 14 |
| 1st_CK_Unagi_NF | CCAGCAGGGAGGTACT ATGCCATTCGGCAATACGCAC | 15 |
| 1st_CK_Unagi_NR_01 | GGCCCGCCCGAGCACTTATTCTA TTTCTGTGCGGGAATCA | 16 |
| 1st_CK_Rabbit_NF | CCAGCAGGGAGGTACT ATGCCGTTCGGCAACACC | 17 |
| 1st_CK_Rabbit_NR_01 | GGCCCGCCCGAGCACTTATTCTA CTTCTGGGCCGGGATCATG | 18 |

FIG. 5 illustrates the results. Note that, for comparison, CK_T (Thunnus) was also put into the lane and subjected to Western blotting. As is clear from FIG. 5, it was confirmed that CK of each biological species is translated from the translation template mRNA by the element contained in the translation reaction liquid, the translated CK constructed an energy regeneration system, and thereby CK self-proliferated also for bovine, which is a mammal, for chicken, which is a bird, and for Thunnus and eel, which are fish. From the above results, it can be said that self-construction of an energy regeneration system is not limited by the biological species.

Synthesis of Protein Other than Energy Regeneration Enzyme

Next, synthesis of proteins by using systems that cause an energy regeneration enzyme to self-proliferate was performed.

### Example 3

Synthesis in which GFP was the target protein was performed by using CK_T (L3 of FIG. 4), which is a translation product obtained by <Example 1> described above. Table 12 lists the composition of translation reaction liquids. For CK_T that is the translation product, an experiment was performed with different addition amounts as listed by Nos. 1 to 4. The target protein was GFP, and the translation template mRNA described in Reference example 1 was used. In the negative control (NC), no CK, CK_T, or mRNA for GFP was contained, and in the positive control (PC), CK was used instead of CK_T that is the translation product. A translation reaction was performed with the composition in Table 11 for about 15 hours, the experiment was performed in the same procedure as <Reference example 1> described above, and the fluorescence intensity of the synthesized GFP was measured. Note that the unit of the numerical values in Table 11 is μL. The stock concentration of the added CK was 20 mg/mL, and the final concentration was 200 ng/μL.

TABLE 12

| | NC | No. 1 | No. 2 | No. 3 | No. 4 | PC |
|---|---|---|---|---|---|---|
| WGE | 20 | 20 | 20 | 20 | 20 | 20 |
| CK | — | — | — | — | — | 2.2 |
| CK_T | — | 0 | 5 | 10 | 15 | — |
| AAM | 40 | 40 | 40 | 40 | 40 | 40 |
| mRNA | 0 | 80 | 80 | 80 | 80 | 80 |
| (GFP) | | | | | | |
| MilliQ | 160 | 80 | 75 | 70 | 65 | 77.8 |
| total | 220 | 220 | 220 | 220 | 220 | 220 |

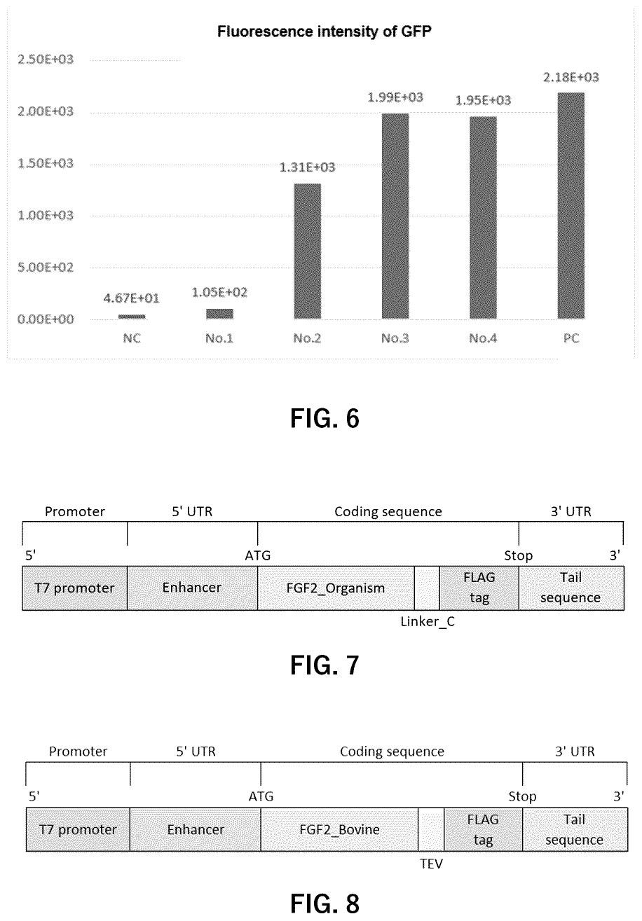

FIG. 6 illustrates the results. While almost no production of GFP was found in No. 1 in which the translation product of CK_T was not put in, production of GFP was observed in No. 2 to No. 4. From the results above, it was confirmed that the protein production method disclosed in the present application can produce the target protein by using CK_T that is the translation product translated from translation template mRNA.

### Example 4

Synthesis in which FGF2 corresponding to each biological species listed in Table 13 below was the target protein was performed by using the CK of bovine, chicken, Thunnus, and eel, which is the translation product obtained in <Example 2> described above.

TABLE 13

| | Protein | | | |
|---|---|---|---|---|
| mark | Name | Acc. No. | Organism | Company |
| FGF2_B | FGF2 | P03969 | Bovine | Eurofins Genomics |
| FGF2_C | FGF2 | NP_990764.1 | Chicken | Eurofins Genomics |
| FGF2_T | FGF2 | XP_042274368.1 | Thunnus | Eurofins Genomics |
| FGF2_U | FGF2 | XP_035281190.1 | Unagi | Eurofins Genomics |

### (1) Transcription Template DNA for FGF2 (Chicken, Thunnus, Eel)

FIG. 7 illustrates the overview of the transcription template DNA for FGF2 of chicken, Thunnus, and eel (hereafter, which may be referred to as “FGF2_DNA”). Further, Table 14 lists the sequences of respective regions of the FGF2_DNA. Note that the FGF2 sequences of Chicken, Thunnus, and Unagi (eel) of above Table 13 are inserted in the field of FGF2_Organism in Table 14 below.

TABLE 14

| Region | Site | DNA Sequence | SEQ.ID |
|---|---|---|---|
| Promoter | T7 promoter | CCCGCGAAAT TAATACGACT CACTATA | 19 |
| 5′UTR | Enhancer | GGGCTCACCT ATCTCTCTAC ACAAAACATT TCCCTACATA CAACTTTCAA | 20 |
| | | CTTCCTATT | |
| Coding | FGF2_Organism | | |
| Sequence | Linker_C | CTC CAG CAG GGA GGT ACT | 21 |
| | FLAG tag | GAC TAC AAG GAT GAC GAT GAC AAG | 22 |
| | Stop codon | TAG | |
| 3′UTR | Tail sequence | AATAAGTGCT CGGGCGGGCC AAAAAAAAAA AAAAA | 23 |

### (2) Transcription Template DNA for FGF2 (Bovine)

FIG. 8 illustrates the overview of the transcription template DNA for FGF2 of bovine. Further, Table 15 lists the sequences of respective regions of the transcription template DNA for the FGF2 of bovine. Note that the FGF2 sequence of Bovine in above Table 13 is inserted in the field of FGF2_Bovine in Table 15 below.

TABLE 15

| Region | Site | DNA Sequence | SEQ.ID |
|---|---|---|---|
| Promoter | T7 promoter | CCCGCGAAAT TAATACGACT CACTATA | 24 |
| 5′UTR | Enhancer | GGGAATTCCC ATCTGCTCCT CCAACTCCAA TCCAATC | 25 |
| Coding | FGF2_Bovine | | |
| Sequence | TEV | GAA AAC CTG TAT TTT CAG AGC | 26 |
| | FLAG tag | GAC TAC AAG GAT GAC GAT GAC AAG | 27 |
| | Stop codon | TAG | |
| 3′UTR | Tail sequence | AATAAGTGCT CGGGCGGGCC AAAAAAAAAA AAAAA | 28 |

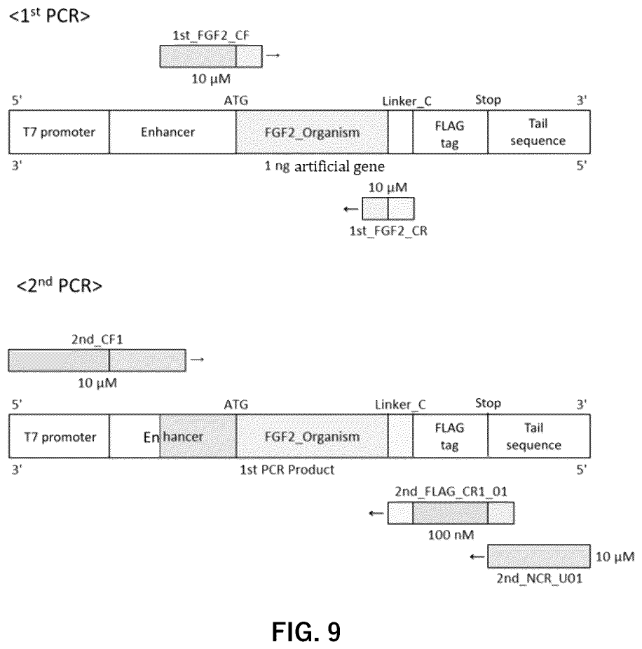

The procedure of preparing FGF2_DNA by PCR will be described with reference to FIG. 9. Note that, although the structure of transcription template DNA in the case of bovine differs from that illustrated in FIG. 9, the procedure is the same. Primers were designed as illustrated in FIG. 9, and FGF2_DNA was prepared by two-step PCR. Table 16 lists the 1st PCR primers of respective biological species, Table 17 lists the 2nd PCR primers of chicken, Thunnus, and eel, and Table 18 lists the 2nd PCR primers of bovine. Note that “2nd_NCF_U01” and “2nd_FLAG_CR1_E15” in Table 18 correspond to “2nd_CF1” and “2nd_FLAG_CR1_01” in FIG. 9, respectively. The “2nd_NCR_U01” is common to all the biological species.

The reaction solution compositions of PCR are the same as those of Table 5 and Table 6 in Example 1, and the PCR program is also the same as that of Table 7 in Example 1. Note that the synthesis method of the transcription template DNA, the reagent, and the machine are also the same as those in Example 1.

TABLE 16

| Primer name | Primer sequence 5′ to 3′ | SEQ.ID |
|---|---|---|
| FGF2_Chicken_CF | CACAAAACATTTCCCTACATACAACTTTCAACTTCCTATT | 29 |
| | ATGCCAGCCCTTCCAGACGATG | |
| FGF2_Chicken_CR | AGTACCTCCCTGCTGGAGACC ACTTTTGGCCGACATGGG | 30 |
| FGF2_Thunnus_CF | CACAAAACATTTCCCTACATACAACTTTCAACTTCCTATT | 31 |
| | ATGGCTACAGGGGAGATAACCAC | |
| FGF2_Thunnus_CR | AGTACCTCCCTGCTGGAGACC GCTCTTAGCCGACATGGG | 32 |
| FGF2_Unagi_CF | CACAAAACATTTCCCTACATACAACTTTCAACTTCCTATT | 33 |
| | ATGCCTCCTATGCCCGAAGATGGTG | |
| FGF2_Unagi_CR | AGTACCTCCCTGCTGGAGACC AGATTTGGCTGACATCGGCAGG | 34 |
| FGF2_Bovine_CF_E15 | CTGCTCCTCCAACTCCAATCCAATC ATGCCAGCCTTACCG | 35 |
| | GAAGATGGG | |
| FGF2_Bovine_CR_E15 | GCTCTGAAAATACAGGTTTTC CGACTTGGCTGACATCGGC | 36 |

TABLE 17
<chicken, Thunnus, eel>

| Primer name | Primer sequence 5′ to 3′ | SEQ.ID. |
|---|---|---|
| 2nd_CF1 | CCCGCGAAAT TAATACGACT CACTATAGGG CTCACCTATC | 37 |
| | TCTCTACACA AAACATTTCC | |
| 2nd_FLAG_CR1_01 | GGCCCGCCCG AGCACTTATT CTACTTGTCA TCGTCATCCT | 38 |
| | TGTAGTCAGT ACCTCCCTGC TGG | |
| 2nd_NCR_U01 | TTTTTTTTTT TTTTTGGCCC GCCCGAGCAC | 39 |

TABLE 18
<bovine>

| Primer name | Primer sequence 5′ to 3′ | SEQ.ID. |
|---|---|---|
| 2nd_NCF_U01 | CCCGCGAAT TAATACGACT CACTATAGGG AATTCCCATC TGCTCCTCCA | 40 |
| | ACTCCAAT | |
| 2nd_FLAG_CR1_E15 | GGCCCGCCCG AGCACTTATT CTACTTGTCA TCGTCATCCT TGTAGTCGCT | 41 |
| | CTGAAAATAC AGGTTTTC | |
| 2nd_NCR_U01 | TTTTTTTTTT TTTTTGGCCC GCCCGAGCAC | 42 |

### (3) Transcription Reaction

A transcription reaction was performed in the same procedure as in Example 1 by using the transcription template DNA for FGF2 (chicken, Thunnus, eel, bovine) described in (1) and (2) above.

### (4) Synthesis of FGF2

Synthesis of FGF2 was performed by using various CK that is the translation product obtained in <Example 2> described above and the translation template mRNA obtained in (3) described above. Table 19 lists the composition of the translation reaction liquid. An experiment was performed in the same procedure as in <Example 1> described above with the composition listed in Table 19. Note that, for the primary antibodies, the following primary antibody was used instead of the primary antibodies in Example 1.
Primary antibody: anti-DYKDDDDK tag (monoclonal antibody Code No. 018-22381; FUJIFILM Wako Pure Chemical Corporation)

TABLE 19

| | FGF2_B | FGF2_C | FGF2_T | FGF2_U |
|---|---|---|---|---|
| WGE | 20 | 20 | 20 | 20 |
| CK | 0 | 0 | 0 | 0 |
| CK_B | 5 | — | — | — |
| CK_C | — | 5 | — | — |
| CK_T | — | — | 5 | — |
| CK_U | — | — | — | 5 |
| AAM | 40 | 40 | 40 | 40 |
| mRNA | Bovine 80 | Chicken 80 | Thunnus 80 | Unagi 80 |
| MilliQ | 75 | 75 | 75 | 75 |
| total | 220 | 220 | 220 | 220 |

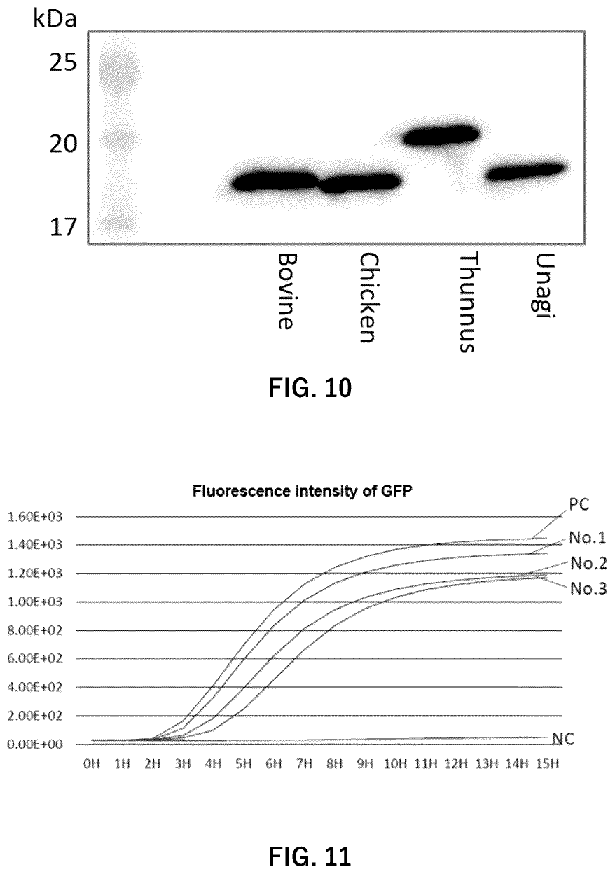

FIG. 10 illustrates the results. As is clear from the results in FIG. 10, it was confirmed that the target protein can be synthesized by using an energy regeneration enzyme derived from the same biological species.

### Co-Expression of Energy Regeneration Enzyme and Target Protein

### Example 5

Next, a translation reaction was performed in a state where the translation template mRNA for translating an energy regeneration enzyme and the translation template mRNA for translating a target protein coexist. The experimental procedure is described below.

### (1) Energy Regeneration Enzyme

The translation template mRNA was prepared in the same procedure as in Example 1 by using CK_R listed as No. 1 of table 2 in Example 1.

### (2) Target Protein

The translation template mRNA for GFP described in Reference example 1 was used.

### (3) Co-Expression of Energy Regeneration Enzyme and Target Protein

Table 20 lists the composition of the translation reaction liquid. In the negative control (NC), no CK, CK_Rabbit, or mRNA for GFP was contained, and in the positive control (PC), CK was used instead of the translation template mRNA for CK Rabbit. A translation reaction was performed with the composition in Table 20 for about 15 hours, the experiment was performed in the same procedure as <Reference Example 1> described above, and the fluorescence intensity of the synthesized GFP was measured.

TABLE 20

| | PC | No. 1 | No. 2 | No. 3 | NC | note |
|---|---|---|---|---|---|---|
| WGE(−CK) | 20 | 20 | 20 | 20 | 20 | |
| AAM | 40 | 40 | 40 | 40 | 40 | |
| CK | 2.2 | — | — | — | — | 20 mg/mL |
| CK_Rabbit | — | 20 | 10 | 10 | — | mRNA |
| GFP | 40 | 20 | 30 | 40 | — | mRNA |
| MilliQ | X | X | X | X | X | |
| total | 220 | 220 | 220 | 220 | 220 | |

FIG. 11 illustrates the results. As is clear from FIG. 11, it was confirmed that, even in a state where the translation template mRNA for translating an energy regeneration enzyme and the translation template mRNA for translating a target protein coexist, the energy regeneration system can be self-constructed, and the target protein can be synthesized.

### INDUSTRIAL APPLICABILITY

According to the protein production method, the cultured meat production method, the additive used for a cultured meat production method, and the kit used for the protein production method disclosed in the present application, an energy regeneration system of a cell-free protein synthetic system can be constructed by using an energy regeneration enzyme translated from translation template mRNA. Therefore, the protein production method, the cultured meat production method, the additive used for a cultured meat production method, and the kit used for the protein production method disclosed in the present application are useful in industries that need cell-free protein synthesis, such as food industries, pharmaceutical industries, or research institutions.

1. A protein production method comprising a protein translation step of translating a protein by using translation template mRNA in the presence of an element for translating translation template mRNA into a protein in the absence of cells,
wherein the translation template mRNA includes translation template mRNA for translating an energy regeneration enzyme, and
wherein no energy regeneration enzyme is added before and at start of protein synthesis, and an energy regeneration system is constructed by using only the energy regeneration enzyme translated from the translation template mRNA for translating the energy regeneration enzyme.

2. The protein production method according to claim 1, wherein the translation template mRNA further includes translation template mRNA for translating a target protein.

3. The protein production method according to claim 2, wherein the protein translation step includes
a first protein translation step of translating the energy regeneration enzyme by using the translation template mRNA for translating the energy regeneration enzyme, and
a second protein translation step of translating the target protein by using a translation product of the first protein translation step and the translation template mRNA for translating the target protein.

4. The protein production method according to claim 2, wherein the protein translation step is performed in a state where the translation template mRNA for translating the energy regeneration enzyme and the translation template mRNA for translating the target protein coexist.

5. The protein production method according to claim 1, wherein the energy regeneration enzyme is at least one type selected from a group consisting of creatine kinase, nucleoside 2-phosphate kinase, arginine kinase, and adenylate kinase.

6. The protein production method according to claim 1, wherein the translation template mRNA further includes translation template mRNA for translating at least one type selected from a group consisting of inorganic diphosphatase, inorganic pyrophosphatase, and disulfide isomerase.

7. The protein production method according to claim 2, wherein the translation template mRNA for translating the energy regeneration enzyme and the translation template mRNA for translating the target protein are translated by
using transcription template DNA including a region to code the translation template mRNA and a promotor region arranged at the 5′ terminal of the region to code the translation template mRNA, and
using the transcription template DNA in the presence of an element for transcribing the transcription template DNA into mRNA in the absence of cells.

8. The protein production method according to claim 2, wherein the energy regeneration enzyme and the target protein are from the same biological species.

9. The protein production method according to claim 2, wherein the target protein is a growth factor.

10. A cultured meat production method comprising a step of culturing cells by using a culture liquid containing a translation product as an additive, the translation product being produced by the protein production method according to claim 9.

11. A kit used for a protein production method, the kit comprising:
translation template mRNA for translating an energy regeneration enzyme; and
an element for translating, into a protein, the translation template mRNA for translating the energy regeneration enzyme in the absence of cells,
wherein an energy regeneration system is constructed by using only the energy regeneration enzyme translated from the translation template mRNA for translating the energy regeneration enzyme, so that no energy regeneration enzyme is added to the kit.

12. The kit according to claim 11 further comprising translation template mRNA for translating a target protein.

13. The kit according to claim 12, wherein the energy regeneration enzyme and the target protein are from the same biological species.

14. The protein production method according to claim 2, wherein the energy regeneration enzyme is at least one type selected from a group consisting of creatine kinase, nucleoside 2-phosphate kinase, arginine kinase, and adenylate kinase.

15. The protein production method according to claim 3, wherein the energy regeneration enzyme is at least one type selected from a group consisting of creatine kinase, nucleoside 2-phosphate kinase, arginine kinase, and adenylate kinase.

16. The protein production method according to claim 4, wherein the energy regeneration enzyme is at least one type selected from a group consisting of creatine kinase, nucleoside 2-phosphate kinase, arginine kinase, and adenylate kinase.

17. The protein production method according to claim 2, wherein the translation template mRNA further includes translation template mRNA for translating at least one type selected from a group consisting of inorganic diphosphatase, inorganic pyrophosphatase, and disulfide isomerase.

18. The protein production method according to claim 3, wherein the translation template mRNA further includes translation template mRNA for translating at least one type selected from a group consisting of inorganic diphosphatase, inorganic pyrophosphatase, and disulfide isomerase.

19. The protein production method according to claim 4, wherein the translation template mRNA further includes translation template mRNA for translating at least one type selected from a group consisting of inorganic diphosphatase, inorganic pyrophosphatase, and disulfide isomerase.

20. The protein production method according to claim 3, wherein the target protein is a growth factor.


SEQUENCE LISTING (42 sequences)

SEQ ID NO: 1 - DNA, 27 bp, synthetic construct
  source 1..27: mol_type = other DNA
cccgcgaaattaatacgactcactata

SEQ ID NO: 2 - DNA, 59 bp, synthetic construct
  source 1..59: mol_type = other DNA
gggctcacctatctctctacacaaaacatttccctacatacaactttcaacttcctatt

SEQ ID NO: 3 - DNA, 27 bp, synthetic construct
  source 1..27: mol_type = other DNA
tatccttatgacgtgcctgactatgcc

SEQ ID NO: 4 - DNA, 18 bp, synthetic construct
  source 1..18: mol_type = other DNA
ctccagcagggaggtact

SEQ ID NO: 5 - DNA, 35 bp, synthetic construct
  source 1..35: mol_type = other DNA
aataagtgctcgggcgggccaaaaaaaaaaaaaaa

SEQ ID NO: 6 - DNA, 35 bp, synthetic construct
  source 1..35: mol_type = other DNA
ccagcagggaggtactatgccctttggtaacacac

SEQ ID NO: 7 - DNA, 45 bp, synthetic construct
  source 1..45: mol_type = other DNA
ggcccgcccgagcacttattctacttttgagcgggtatcatagag

SEQ ID NO: 8 - DNA, 122 bp, synthetic construct
  source 1..122: mol_type = other DNA
cgactcactatagggctcacctatctctctacacaaaacatttccctacatacaactttcaacttcctattatgtatccttatgacgtgcctgactatgccctccagcagggaggtactatg

SEQ ID NO: 9 - DNA, 28 bp, synthetic construct
  source 1..28: mol_type = other DNA
cccgcgaaattaatacgactcactatag

SEQ ID NO: 10 - DNA, 30 bp, synthetic construct
  source 1..30: mol_type = other DNA
tttttttttttttttggcccgcccgagcac

SEQ ID NO: 11 - DNA, 40 bp, synthetic construct
  source 1..40: mol_type = other DNA
ccagcagggaggtactatgccctttggcaatacccataac

SEQ ID NO: 12 - DNA, 40 bp, synthetic construct
  source 1..40: mol_type = other DNA
ggcccgcccgagcacttattctacttttgggctgggatca

SEQ ID NO: 13 - DNA, 35 bp, synthetic construct
  source 1..35: mol_type = other DNA
ccagcagggaggtactatgcccttctccagcacac

SEQ ID NO: 14 - DNA, 40 bp, synthetic construct
  source 1..40: mol_type = other DNA
ggcccgcccgagcacttattctacttttgcgctggaatca

SEQ ID NO: 15 - DNA, 37 bp, synthetic construct
  source 1..37: mol_type = other DNA
ccagcagggaggtactatgccattcggcaatacgcac

SEQ ID NO: 16 - DNA, 40 bp, synthetic construct
  source 1..40: mol_type = other DNA
ggcccgcccgagcacttattctatttctgtgcgggaatca

SEQ ID NO: 17 - DNA, 34 bp, synthetic construct
  source 1..34: mol_type = other DNA
ccagcagggaggtactatgccgttcggcaacacc

SEQ ID NO: 18 - DNA, 42 bp, synthetic construct
  source 1..42: mol_type = other DNA
ggcccgcccgagcacttattctacttctgggccgggatcatg

SEQ ID NO: 19 - DNA, 27 bp, synthetic construct
  source 1..27: mol_type = other DNA
cccgcgaaattaatacgactcactata

SEQ ID NO: 20 - DNA, 59 bp, synthetic construct; identical to SEQ ID NO: 2
  source 1..59: mol_type = other DNA

SEQ ID NO: 21 - DNA, 18 bp, synthetic construct
  source 1..18: mol_type = other DNA
ctccagcagggaggtact

SEQ ID NO: 22 - DNA, 24 bp, synthetic construct
  source 1..24: mol_type = other DNA
gactacaaggatgacgatgacaag

SEQ ID NO: 23 - DNA, 35 bp, synthetic construct; identical to SEQ ID NO: 5
  source 1..35: mol_type = other DNA

SEQ ID NO: 24 - DNA, 27 bp, synthetic construct
  source 1..27: mol_type = other DNA
cccgcgaaattaatacgactcactata

SEQ ID NO: 25 - DNA, 37 bp, synthetic construct
  source 1..37: mol_type = other DNA
gggaattcccatctgctcctccaactccaatccaatc

SEQ ID NO: 26 - DNA, 21 bp, synthetic construct
  source 1..21: mol_type = other DNA
gaaaacctgtattttcagagc

SEQ ID NO: 27 - DNA, 24 bp, synthetic construct
  source 1..24: mol_type = other DNA
gactacaaggatgacgatgacaag

SEQ ID NO: 28 - DNA, 35 bp, synthetic construct; identical to SEQ ID NO: 5
  source 1..35: mol_type = other DNA

SEQ ID NO: 29 - DNA, 62 bp, synthetic construct
  source 1..62: mol_type = other DNA
cacaaaacatttccctacatacaactttcaacttcctattatgccagcccttccagacgatg

SEQ ID NO: 30 - DNA, 39 bp, synthetic construct
  source 1..39: mol_type = other DNA
agtacctccctgctggagaccacttttggccgacatggg

SEQ ID NO: 31 - DNA, 63 bp, synthetic construct
  source 1..63: mol_type = other DNA
cacaaaacatttccctacatacaactttcaacttcctattatggctacaggggagataaccac

SEQ ID NO: 32 - DNA, 39 bp, synthetic construct
  source 1..39: mol_type = other DNA
agtacctccctgctggagaccgctcttagccgacatggg

SEQ ID NO: 33 - DNA, 65 bp, synthetic construct
  source 1..65: mol_type = other DNA
cacaaaacatttccctacatacaactttcaacttcctattatgcctcctatgcccgaagatggtg

SEQ ID NO: 34 - DNA, 43 bp, synthetic construct
  source 1..43: mol_type = other DNA
agtacctccctgctggagaccagatttggctgacatcggcagg

SEQ ID NO: 35 - DNA, 49 bp, synthetic construct
  source 1..49: mol_type = other DNA
ctgctcctccaactccaatccaatcatgccagccttaccggaagatggg

SEQ ID NO: 36 - DNA, 40 bp, synthetic construct
  source 1..40: mol_type = other DNA
gctctgaaaatacaggttttccgacttggctgacatcggc

SEQ ID NO: 37 - DNA, 60 bp, synthetic construct
  source 1..60: mol_type = other DNA
cccgcgaaattaatacgactcactatagggctcacctatctctctacacaaaacatttcc

SEQ ID NO: 38 - DNA, 63 bp, synthetic construct
  source 1..63: mol_type = other DNA
ggcccgcccgagcacttattctacttgtcatcgtcatccttgtagtcagtacctccctgctgg

SEQ ID NO: 39 - DNA, 30 bp, synthetic construct; identical to SEQ ID NO: 10
  source 1..30: mol_type = other DNA

SEQ ID NO: 40 - DNA, 58 bp, synthetic construct
  source 1..58: mol_type = other DNA
cccgcgaaattaatacgactcactatagggaattcccatctgctcctccaactccaat

SEQ ID NO: 41 - DNA, 68 bp, synthetic construct
  source 1..68: mol_type = other DNA
ggcccgcccgagcacttattctacttgtcatcgtcatccttgtagtcgctctgaaaatacaggttttc

SEQ ID NO: 42 - DNA, 30 bp, synthetic construct; identical to SEQ ID NO: 10
  source 1..30: mol_type = other DNA